(19) United States
(12) Patent Application Publication
(10) Pub. No.: US 2025/0316365 A1
(43) Pub. Date: Oct. 9, 2025

(54) SYSTEMS AND METHODS FOR CONTENT AWARE USER INTERFACE OVERLAYS

(71) Applicant: INTUITIVE SURGICAL OPERATIONS, INC., Sunnyvale, CA (US)

(72) Inventors: Simon P. Dimaio, San Carlos, CA (US); Brandon D. Itkowitz, San Jose, CA (US)

(21) Appl. No.: 18/865,277

(22) PCT Filed: May 10, 2023
(86) PCT No.: PCT/US2023/021636

§ 371 Date: Nov. 12, 2024

Related U.S. Application Data
(60) Provisional application No. 63/341,881, filed on May 13, 2022.

(51) Int. Cl. G16H 30/40; G06F 3/0482; G06T 5/50; G06T 7/12; G06T 7/13

(52) U.S. Cl. CPC G16H 30/40; G06F 3/0482; G06T 5/50; G06T 7/12; G06T 7/13; G06T 2207/20221

(57) ABSTRACT

A medical system may comprise a display system for displaying a display area and a control system. The control system may include a processing unit including one or more processors. The processing unit may be configured to generate an image of a field of view for display in the display area, generate a user interface component for display in the display area, determine a priority record for an element in the image of the field of view, determine a display characteristic for the user interface component based on the priority record of the element in the field of view, and display the user interface component in accordance with the display characteristic in the display area overlayed on a displayed image of the field of view.

### CROSS-REFERENCED APPLICATIONS

This application claims priority to and benefit of U.S. Provisional Application No. 63/341,881, filed May 13, 2022 and entitled “Systems and Methods for Content Aware User Interface Overlays,” which is incorporated by reference herein in its entirety.

### FIELD

The present disclosure is directed to systems and methods for use in robot-assisted medical procedures, and more particularly to systems and methods for arranging user interface overlays based on an awareness of the content of the underlying field of view images.

### BACKGROUND

Minimally invasive medical techniques are intended to reduce the amount of extraneous tissue that is damaged during diagnostic or surgical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. Minimally invasive medical tools include instruments such as therapeutic instruments, diagnostic instruments, and surgical instruments. Minimally invasive medical tools may also include imaging instruments such as endoscopic instruments that provide a user with an image of a field of view within the patient anatomy.

Some minimally invasive medical tools may be robot-assisted including teleoperated, remotely operated, or otherwise computer-assisted. During a medical procedure, the clinician may view an image of a field of view of the patient anatomy that may include one or more of the minimally invasive medical tools. Various user interface components may be overlayed on the image of the field of view to provide, for example, information, interactive capabilities, and tool controls. Improved systems and methods are needed to present overlayed user interface components based on an awareness of the underlying contents of the field of view.

### SUMMARY

The embodiments of the invention are best summarized by the claims that follow the description.

In one example embodiment, a medical system may comprise a display system for displaying a display area and a control system. The control system may include a processing unit including one or more processors. The processing unit may be configured to generate an image of a field of view for display in the display area, generate a user interface component for display in the display area, determine a priority record for an element in the image of the field of view, determine a display characteristic for the user interface component based on the priority record of the element in the field of view, and display the user interface component in accordance with the display characteristic in the display area overlayed on a displayed image of the field of view.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a flowchart illustrating a method for displaying user interface component overlays based on a system awareness of the contents of the field of view, according to some examples.

FIG. 2 illustrates a graphical user interface including an image of a field of view with an overlayed user interface component, according to some examples.

FIG. 3A illustrates a graphical user interface including an adjusted image of the field of view of FIG. 2 with the overlayed user interface component over a high priority element in the field of view, according to some examples.

FIG. 3B illustrates a graphical user interface including the image of the field of view of FIG. 3A with the overlayed user interface component moved to overlay an element in the field of view with a lower priority.

FIG. 4A illustrates a graphical user interface with the overlayed user interface component over a high priority element in the field of view, according to some examples.

FIG. 4B illustrates a graphical user interface including the field of view of FIG. 4A with the overlayed user interface component moved to overlay an element in the field of view with a lower priority.

FIG. 5A illustrates a graphical user interface including an image of a field of view with an overlayed user interface component, according to some examples.

FIG. 5B illustrates a graphical user interface including an adjusted image of the field of view of FIG. 5A with the overlayed user interface component over a high priority element in the field of view.

FIG. 5C illustrates a graphical user interface including the field of view of FIG. 5B with the overlayed user interface component moved to overlay an element in the field of view with a lower priority.

FIG. 6 illustrates a graphical user interface with layered user interface components, according to some examples.

FIG. 7 illustrates a graphical user interface with an overlayed semi-transparent user interface component including an auxiliary ultrasound image, according to some examples.

FIG. 8 illustrates a graphical user interface with an overlayed user interface component and with accentuated borders outlining the underlying element in the field of view image, according to some examples.

FIG. 9 illustrates a schematic view of a medical system, according to some examples.

FIG. 10 is a perspective view of a manipulator assembly of the medical system of FIG. 9, according to some examples.

FIG. 11 is a front elevation view of an operator's console in a robot-assisted medical system, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In robot-assisted medical procedures, endoscopic images of the surgical environment may provide a clinician with an image of a field of view of the patient anatomy and any medical tools located in the patient anatomy. Various user interface components may be overlayed on the field of view image. User interface components may include, for example, menus, alerts, user messages, indicators, digital tools, and auxiliary images. These overlayed components may occlude portions of the field of view. The presentation of these user interface components may be adjusted, based on an awareness of the image content, to prioritize the display of regions of the image of field of view that are most relevant to the user.

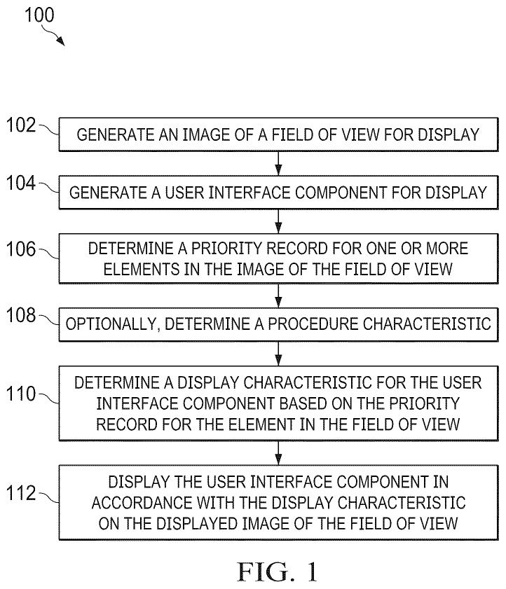

FIG. 1 is a flowchart illustrating a method 100 for displaying user interface component overlays based on an awareness of the contents of the image of the field of view. The methods described herein are illustrated as a set of operations or processes and are described with continuing reference to the additional figures. Not all of the illustrated processes may be performed in all embodiments of the methods. Additionally, one or more processes that are not expressly illustrated in may be included before, after, in between, or as part of the illustrated processes. In some embodiments, one or more of the illustrated processes may be omitted. In some embodiments, one or more of the processes may be implemented, at least in part, in the form of executable code stored on non-transitory, tangible, machine-readable media that when run by one or more processors (e.g., the processing units of a control system such as control system 320) may cause the one or more processors to perform one or more of the processes. In one or more embodiments, the processes may be performed by a control system.

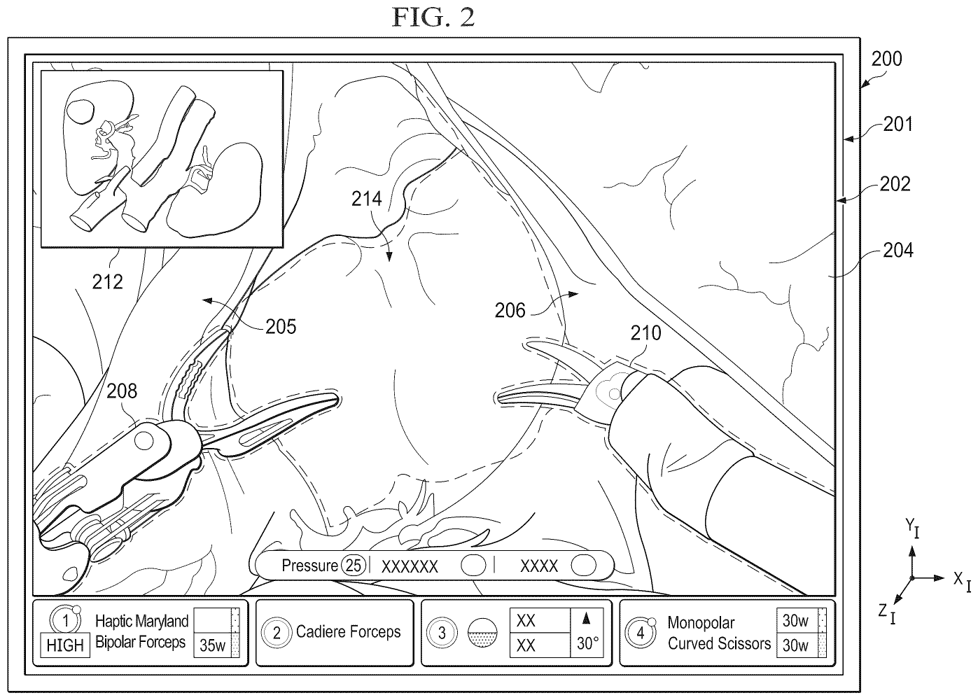

At a process 102, an image of a field of view may be generated for display. FIG. 2, for example, provides a display system 200 including a display area 201 for displaying a graphical user interface 202 including an image 204 of a field of view. The field of view image 204 may be provided by an imaging instrument (e.g., the endoscopic imaging system 715 described below) within an anatomic environment in a patient anatomy. In this example, the image 204 may be a three-dimensional, stereoscopic image, but in other examples, the image may be a two-dimensional image. The image 204 may be an intra-operative, real-time video endoscopic image or may be an image recorded previously, including preoperatively. The image 204 of the field of view may have an image frame of reference X_{I}, Y_{I}, Z_{I} based, for example, on the distal end of the endoscopic imaging system.

Referring again to method 100 of FIG. 1, at a process 104, a user interface component may be generated for display. The user interface component may include a component of the graphical user interface such as a window, alphanumeric text, marker, or other artificial graphical element overlayed on the field of view image or inserted at a three-dimensional position in the field of view image. For example the user interface component may include menus, alerts, user messages, indicators, digital tools, measurement tools, and auxiliary still or video images. Additionally or alternatively, the user interface component may be a digitally modified region of the field of view image. For example, endoscopic images of patient tissue may be modified to enhance the appearance of the vasculature by providing a higher contrast, to amplify local motion of the structures in the endoscopic image, or to blend a fluoroscopic signal with the endoscopic image. FIG. 2, provides an example of a user interface component 212, which in this example may be a window displaying a three-dimensional model of the patient anatomy generated from a pre-operative CT image. The model may be a still three-dimensional image or may be an interactive element that may be manipulated (e.g., rotated) in response to user interaction.

Referring again to FIG. 1, at a process 106, a priority record may be determined for an element in the image of the field of view. Elements in the field of view may include anatomic structures, medical structures such as instruments or other tools, or regions of activity such as moving or bleeding regions. Knowledge about the relative importance, criticality, or relevance of the elements in the image of the field of view may be captured as one or more priority records associated with the elements in the field of view. The priority record may provide a ranking indicator or other indication of relative importance of the element to the clinician viewing the image of the field of view or to the safety or efficacy of the procedure. A relatively high priority record may indicate that the element is a critical structure that the user interface component should avoid occluding. A mapping of the elements in the field of view and their priority records may be stored in a memory (e.g., memory 734) and accessed by a control system (e.g. control system 320) of a robot-assisted medical system (e.g., system 310).

The process 106 may include distinguishing and/or identifying the elements in the image of the field of view and may assigning a priority record to one or more of identified elements. FIG. 2 illustrates identified elements in the field of view image 204 including a tool 208, a tool 210, surgically exposed tissue 214, and surgically uninvolved tissue 205, 206. In some examples, the identified elements in the field of view image may be demarcated by lines (e.g. by dashed lines as shown in FIG. 2), color modification, markers, or other graphical indicators. In other examples, the identified elements may be distinguished by the control system without visible or displayed demarcation. A priority record may be assigned to each of the distinguished elements based on predetermined prioritization categories for various types of structures. A prioritization scale may also be predefined. For example, a scale of 1 (low criticality or relevance) to 5 (high criticality or relevance) may be used to assign priority records based on prioritization categories such as instruments under clinician control, instruments not under clinician control, surgically involved tissue structures, surgically uninvolved tissue structures, regions of tissue motion (e.g. beating, pulsating, moved by an instrument), or regions of fluid flow (e.g. bleeding, mucous flow). In this example of FIG. 2, the activated tools 208, 210 may be assigned a priority record of 5 (high criticality or relevance), the exposed tissue 214 as surgically involved tissue structure may be assigned a priority record of 3 (medium criticality or relevance), and the surgically uninvolved tissue regions 205, 206 may be assigned a prior record of 1 (low criticality or relevance). Any of a variety of linear scales, multi-dimensional parameters, or a composite of ranking and factors may be used to determine relative priority records for the elements in the image of the field of view. Determinations of criticality and relevance may be based on patient health and safety, operator interest, significance to the medical procedure or other criteria.

In some examples, a priority record may be assigned to an element in the image of the field of view by a clinician or other operator viewing the display, such as a surgeon at a surgeon console or a laparoscopic tool operator at a patient bedside. An operator priority record assignment may include identifying the elements in the image of the field of view by marking, outlining, providing a region boundary, annotating, or selecting from a menu of image-segmented elements. The priority record may be assigned to operator-identified elements. In some examples, the image of the field of view may be graphically segmented (e.g., into pixels or voxels) and a priority record may be assigned to regions of the segmented image based on computer vision or a computer image analysis of the image and predetermined categories of elements detected by the image analysis. The analysis may be conducted using artificial intelligence. The image analysis may detect tissue structures, tissue regions, tools, anatomic landmarks, tool or tissue motion, anatomic motion, or any changes in tools or tissue structures and may determine the relevance of the structures to identify which should not be obscured from view. In some examples, and image analysis may assign priority records based on co-registered pre-operative models including identified and prioritized model elements, co-registered intra-operative imaging modalities (e.g. ultrasound, fluorescence imaging), or physical fiducials or other markers in the image of the field of view. In some examples, the priority records may be generated and assigned by a control system or other processor-based system and may be modified or supplemented by a clinician or other operator.

In some examples, a priority record may be dynamic. For example, the priority record for a tool element may change from having a low priority record when it is detected to be inactivated to a higher priority record when activation is detected. The priority record for a region of tissue may change from a low priority record when it is detected as uninvolved in the medical procedure to a higher priority when it is detected as being adjacent to an activated tool. A tissue element may also change to a higher priority when exposed, moved, manipulated by a tool or when changing state, such as from a non-bleeding to a bleeding state. Some regions, areas, or elements of the field of view may not be assigned a priority record.

Referring again to FIG. 1, at a process 108 a procedure characteristic may, optionally, be determined. The procedure characteristic may include a procedure type or a step or task within the procedure. The procedure characteristic may include, for example, a type of task being performed in the anatomic environment, such as dissection, suturing, stapling, irrigation, or tissue retraction. The type of task may be used, for example, to determine a location to display user interface components so as not to interfere with the operators view of the task. The procedure characteristic may also include user preferences for the operator stored in a user profile. User preferences may include, for example, the operator's preferred location in the display to view user interface components.

At a process 110, a display characteristic may be determined for the user interface component. The display characteristic may include one or more properties associated with the user interface component such as placement location, placement orientation, minimum size, maximum size, nominal size, transparency, scaling factor, duration of display, and/or intermittence of display. The display characteristic may be static or may be dynamic, responsive to changes in the field of view. The display characteristic for a user interface component may be determined based on the priority records of the elements in the field of view to avoid obtrusive interference by the user interface component. For example, the display characteristic may be the placement location for the user interface component in the graphical user interface to avoid obstructing field of view elements with relatively high priority records and instead to overlay an area with no priority record or a relatively low priority record. The display characteristic may also or alternatively be determined based on the procedure characteristic. For example, the display characteristic may be determined based on the type of task or the user's preference. At a process 112 the user interface component may be displayed, in accordance with the conditions of the display characteristic, on the displayed image of the field of view.

FIGS. 2-8 illustrate examples of implementations of the method 100. With reference to FIG. 2, in the field of view image 204, the activated tools 208, 210 may be assigned a priority record of 5 (high criticality), the exposed tissue 214 may be assigned a priority record of 3 (medium criticality), and the surgically uninvolved tissue regions 205, 206 may be assigned a priority record of 1 (low criticality). The display characteristic for the user interface component 212 may be the display location of the user interface component relative to the field of view image 204. Based on the priority records for the elements in the field of view image 204, the display characteristic may be a display location overlaying the tissue element 205 with a low priority record. If the field of view includes multiple low priority record elements, the display location may be further determined based upon a proximity to higher priority elements, based on operator preference (e.g., prefers overlays to the left/right or top/bottom on the display), or based on other factors.

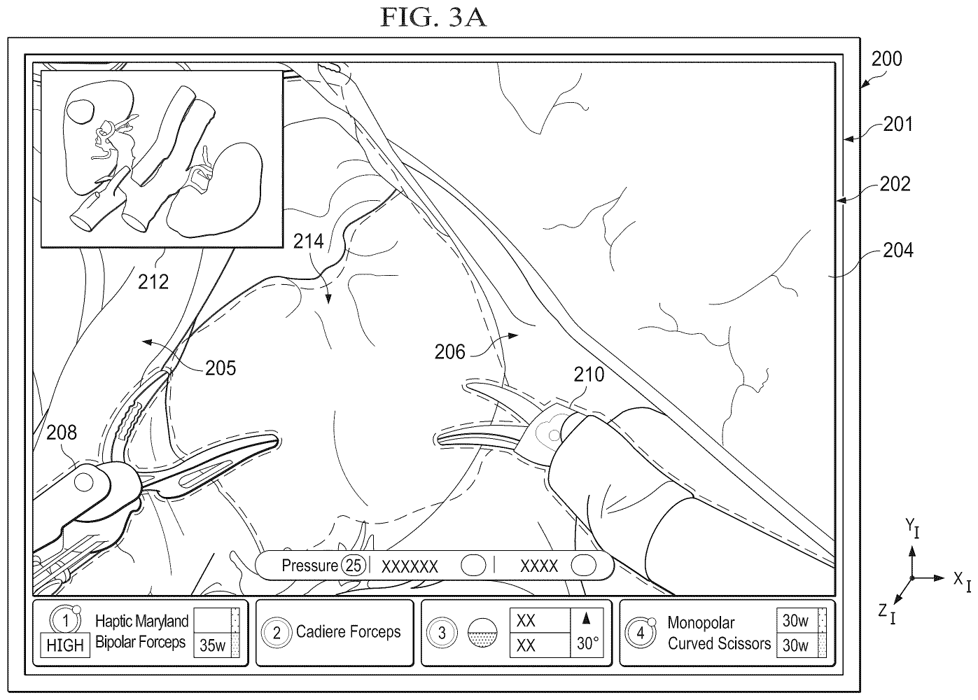

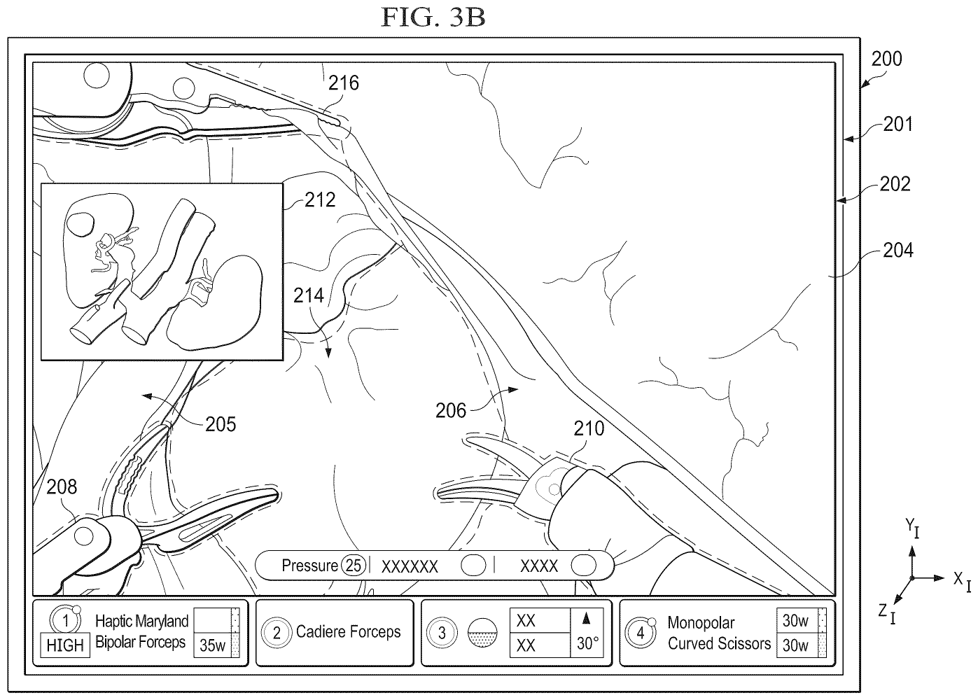

With reference to FIG. 3A, the endoscopic imaging instrument may be panned upward, as compared to FIG. 2, changing the field of view image 204 displayed on the display system 200. The user interface component 212 is in the same location relative to the display system 200 as in FIG. 2, but now occludes the view of an element 216 in the field of view. Element 216 may be an active instrument assigned a priority record of 5 (high criticality). A new display characteristic for the user interface component 212 may be determined based on the priority record for the element 216 now present in the modified field of view image 204. As shown in FIG. 3B, a new display characteristic for the user interface component 212 may be a new display location, relative to the display system 200, that overlays the tissue element 205. In some examples the new display location may be unchanged relative to the tissue element 205 but in other examples it may be changed also relative to the tissue element 205. With the new display characteristic, the user interface component 212 is no longer overlaid on the element 216 having the high priority record. Thus, the elements in the image of the field of view with high priority records remain visible to the clinician. The new display characteristic may be determined and updated dynamically by the control system, as the image analysis determines the change in the field of view or endoscopic imaging instrument. The change in the image of the field of view may be determined from kinematic information related to the endoscopic imaging system or may be determined from image analysis of the image of the field of view. Optionally, the display characteristic for the user interface component may include a nominal size and to maintain the nominal size, the display location for the user interface component 212 may overlap a higher priority element (e.g., element 214). The permitted overlap area may be less than a threshold percentage of the visible area of the element 214 in the field of view image 204.

In some examples, the display characteristic for the user interface component 212 may be a location of the user interface element relative to (e.g., pinned to or tethered to) an element visible in the field of view. For example, the display characteristic for the user interface component 212 may be a display location relative to the tissue element 205. In this example, as long as the tissue element 205 is visible in the field of view image 204, the user interface component 212 is displayed in the same relative position and orientation with respect to the tissue element 205, regardless of the location of the tissue element 205 relative to the display system 200.

In some alternative examples, the display characteristic for the user interface component may be a duty cycle for intermittent display of the user interface component. For example, the user interface component 212 located as in FIG. 3A obstructing the tool element 216 or as in FIG. 3B overlaying the tissue element 205, may also include a display characteristic that causes the user interface component 212 to blink or otherwise display on an intermittent duty cycle so that the underlying element is visible to the viewing clinician during a portion of the duty cycle.

In some alternative examples, the display characteristic for the user interface component may be a duration for display of the user interface component. For example, the user interface component 212 located as in FIG. 3A obstructing the tool element 216 or as in FIG. 3B overlaying the tissue element 205, may also include a display characteristic that causes the user interface component 212 to display for a finite duration of time before the user interface component 212 disappears from the display. In some examples, the user may selectively dismiss the user interface component, causing it to disappear from the display.

In some examples, the display characteristic may allow a display location of the user interface component overlayed on elements with a predetermined range of allowed priority records (e.g., ranked 1-3) and may restrict display locations overlying elements with a predetermined restricted range of allowed priority records (e.g., ranked 4-5).

In some examples, the display characteristic may indicate that the user interface component is occluding an important underlaying object or structure in the field of view or user interface. For example, the display characteristic may include an outline with a distinctive color, line style, icon, or other visual treatment to warn the user of the occlusion.

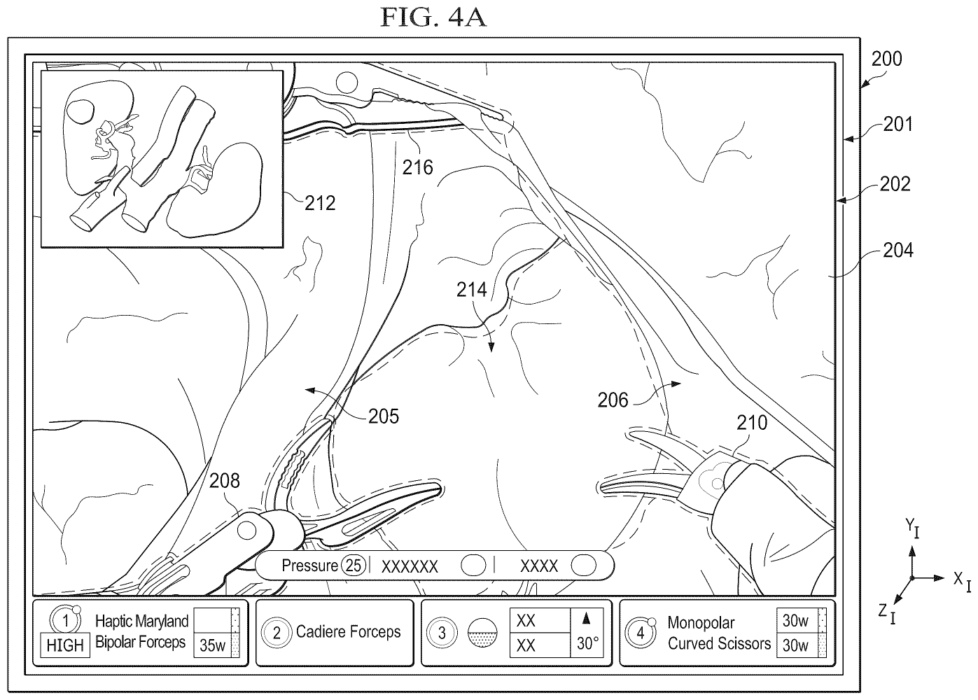

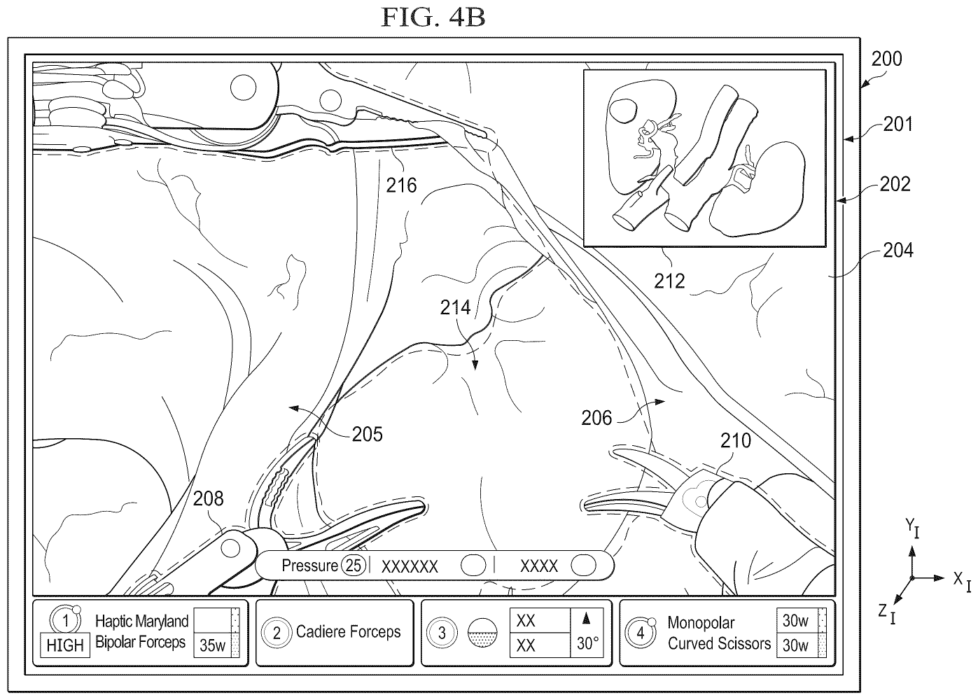

With reference to FIG. 4A, the endoscopic imaging instrument generating the image of the field of view may be zoomed in and panned upward and to the left, as compared to FIG. 2, changing the field of view image 204 displayed on the display system 200. The user interface component 212 is in the same location relative to the display system 200 as in FIG. 2, but now occludes the view of an element 216 in the field of view. Element 216 may be an active instrument assigned a priority record of 5 (high criticality). A new display characteristic for the user interface component 212 may be determined based on the priority record for the element 216 now present in the modified field of view image 204. As shown in FIG. 4B, a new display characteristic for the user interface component 212 may be a new display location, relative to the display system 200, that overlays the tissue element 206 (having a prior record of 1 (low criticality). With the new display characteristic, the user interface component 212 is no longer overlaid on the element 216 having the high priority record. Optionally, the display characteristic for the user interface component may include a scaling factor to maintain a nominal size of the user interface component relative to the display system 200. Thus, in FIG. 4B, even though the elements in the field of view image 204 appear larger than in FIG. 2 due to the imaging instrument zoom factor, the user interface component 212 has not scaled larger in size with the elements in the field of view but rather has stayed the same size as in FIG. 2, relative to the display system 200. In alternative examples, the display characteristic may be a scaling factor that scales proportional to the instrument zoom factor.

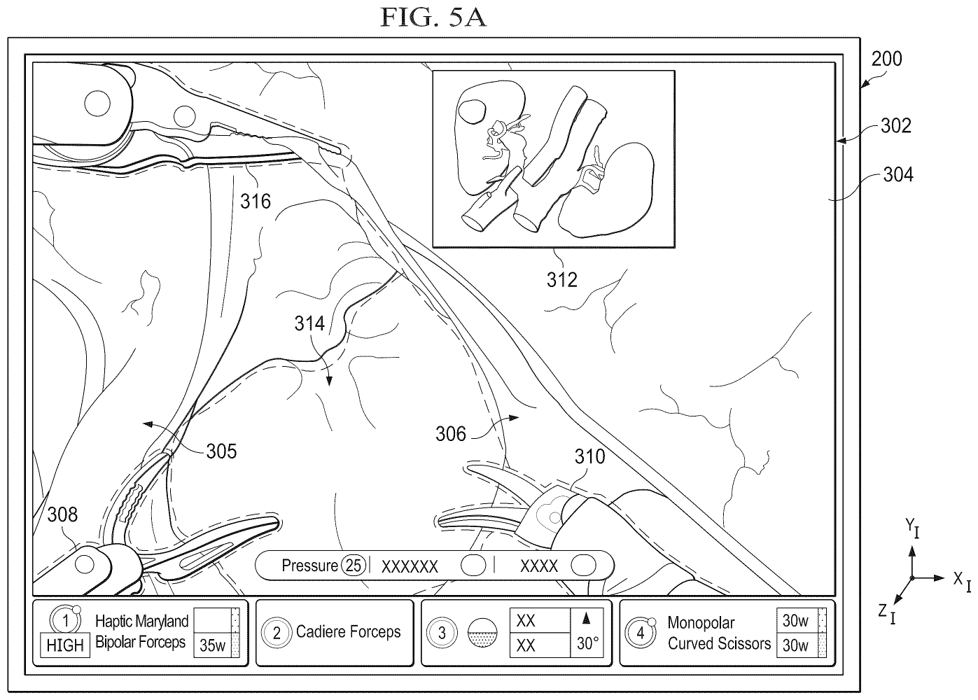

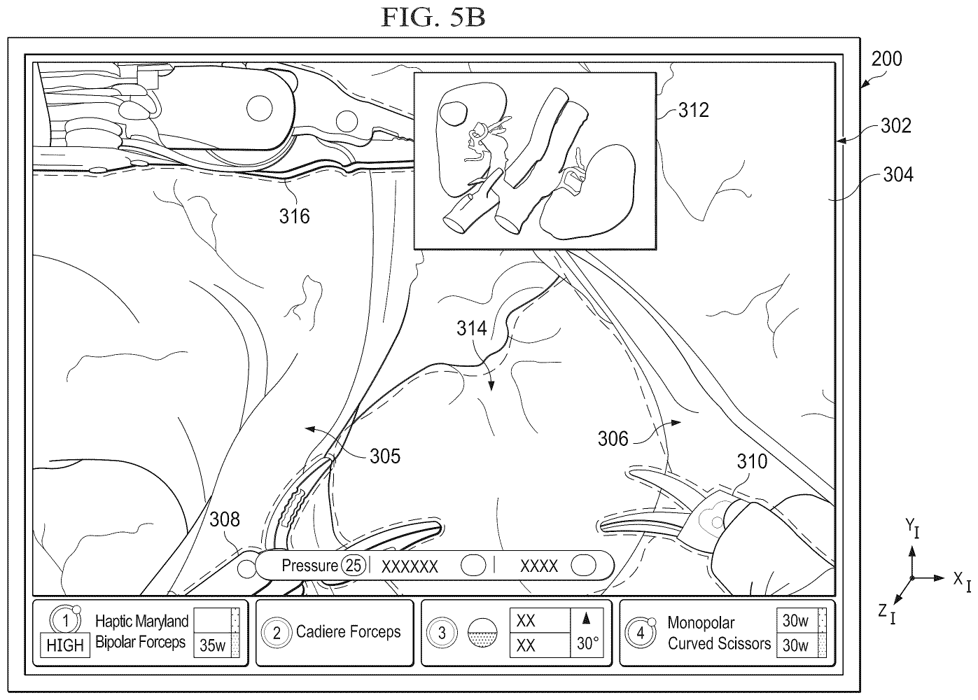

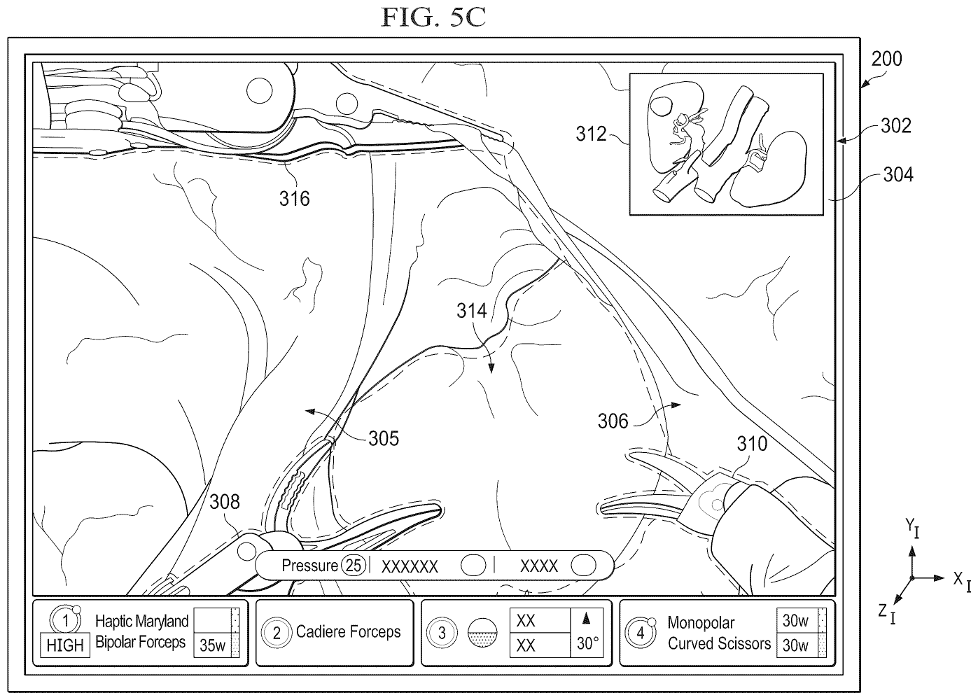

FIG. 5A illustrates the display system 200 displaying a graphical user interface 302 including an image 304 of a field of view. The image 304 includes a tool 308, a tool 310, a tool 316, surgically exposed tissue 314, and surgically uninvolved tissue 305, 306. The tools 308, 310, 316 may be assigned a priority record of 5 (high criticality), the exposed tissue 314 may be assigned a priority record of 3 (medium criticality), and the surgically uninvolved tissue regions 305, 306 may be assigned a priority record of 1 (low criticality). With reference to FIG. 5B, the endoscopic imaging instrument may be panned left, as compared to FIG. 5A, changing the field of view image 304 displayed on the display system 200. The user interface component 312 is in the same location relative to the display system 200 as in FIG. 5A, but now occludes the view of a tool element 316 in the field of view. Tool element 316 may be an active instrument assigned a priority record of 5 (high criticality). A new display characteristic for the user interface component 312 may be determined based on the priority record for the tool element 316 now present in the modified field of view image 304. As shown in FIG. 5C, a new display characteristic for the user interface component 312 may include a new display location, relative to the display system 200, that overlays the tissue element 306. With the new display characteristic, the user interface component 312 is no longer overlaid on the tool element 316 having the high priority record. The new display characteristic for the user interface component 312 may also include a new, smaller scale relative to the display system 200. The scale display characteristic may be determined based upon the area of the tissue element 306 and optionally a minimum size display characteristic for the user interface component 312 that prevents the user interface component from becoming too small.

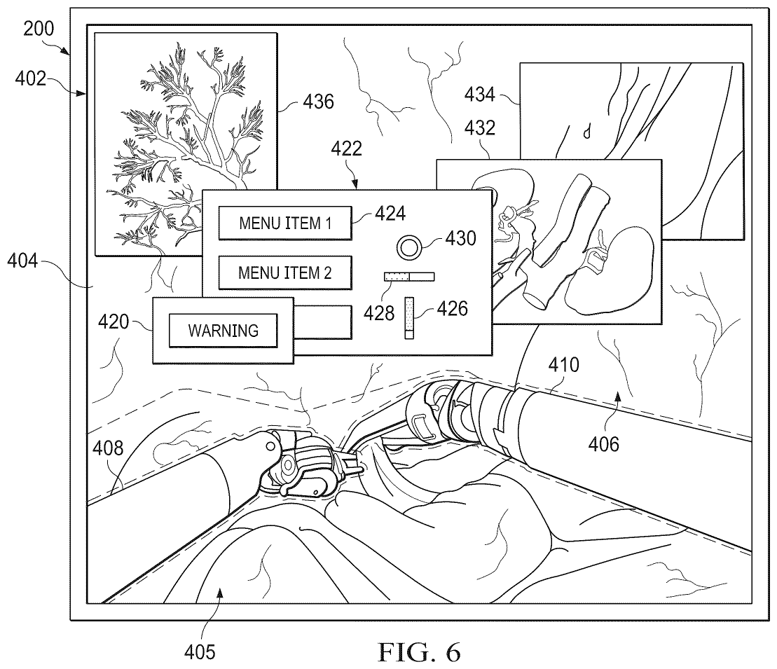

FIG. 6 illustrates the display system 200 displaying a graphical user interface 402 including an image 404 of a field of view. The image 404 includes a tool element 408, a tool element 410, surgically involved tissue element 405, and surgically uninvolved tissue element 406. The tissue element 406 may be assigned a low priority record. In this example, a plurality of user interface components may include a display characteristic that includes a display location overlaid on the tissue element 406. For example, user interface component 420 is a message containing alphanumeric text to communicate information to a clinician during a medical procedure. A user interface component 422 is a window bounding a plurality of user interface components including a menu 424, an indicator 426, a measurement tool 428, and an interactive tool 430. A user interface component 432 includes an interactive anatomic model. A user interface component 434 includes a fluoroscopic anatomic image of the patient anatomy. A user interface component 436 includes a high contrast image of anatomic vasculature. In some examples, the display characteristic for each user interface component also includes a dynamic layer, order, or depth assignment parameter that is used to determine the order of sequential layering of the user interface components. This display characteristic may be useful, for example, if there is insufficient area for the tissue element 406 to fit all of the user interface components concurrently, in a single visible layer. In this example, the user interface component 420 may be assigned a dynamic layer parameter with a value higher than dynamic layer parameters for the user interface components 422-436. As a result, the user interface component 420 may be layered over the other user interface components 422-436, thus allowing the contents of the user interface component 420 to be fully visible to the viewing clinician.

The dynamic layer parameter may change based on the current state of the medical procedure or based on operator selection. For example, a user interface component 420 including a warning message alerting the clinician to an extracorporeal instrument collision may be assigned a high dynamic layer parameter causing the user interface component 420 to dynamically appear and be presented as a top layer, above other user interface components overlaying the tissue element 406. When the instrument collision is rectified, the user interface component 420 may be assigned a lower dynamic layer parameter so that the message window may drop to a lower layer or be closed, allowing another of the user interface components with a higher dynamic layer parameter to be displayed at the top layer. In other examples, if image analysis of the image of the field of view detects a color change in the image that may be associated with patient bleeding, user interface component comprising a warning may be displayed above other user interface components and any displayed user interface components may change display locations so that the area of color change (now assigned a high priority record) is unobstructed by the user interface components.

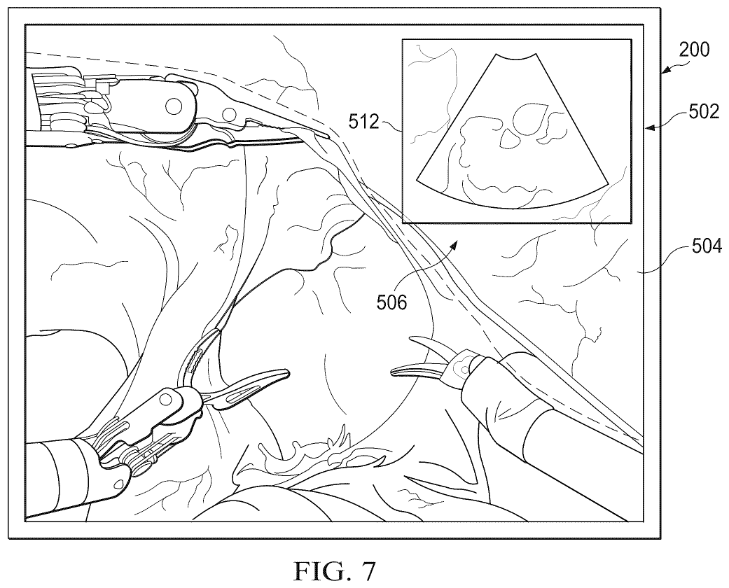

FIG. 7 illustrates the display system 200 displaying a graphical user interface 502 including an image 504 of a field of view. The image 504 includes a surgically uninvolved tissue element 506 that may be assigned a low priority record. In this example, a user interface component 512 may include a window displaying an ultrasound image of the patient anatomy and may include a display characteristic that includes a display distortion such as a display transparency or display blurring for the window when overlaid on the tissue element 506. The display transparency may be fixed level of semi-transparency such as 25% transparency, 50% transparency, 75% transparency, or any transparency level between 0 and 100% that may allow the viewing clinician to at least partially observe the obstructed tissue element 506 underlying the user interface component 512. In some examples, a user interface component 512 with a display transparency may be used to overlay field of view elements with relatively high priority records (e.g. greater than 3 on a 1-5 scale) so that the user interface component may be visualized beneath the user interface component.

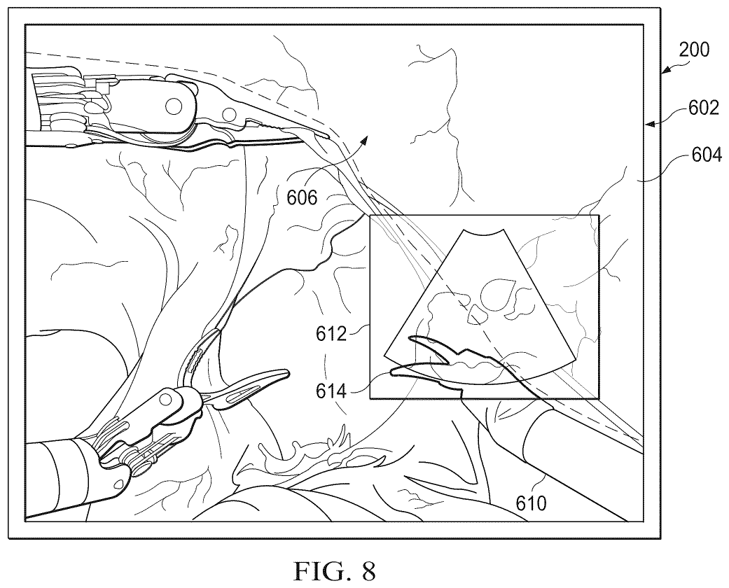

FIG. 8 illustrates the display system 200 displaying a graphical user interface 602 including an image 604 of a field of view. The image 604 includes a surgically uninvolved tissue element 606 that may be assigned a low priority record and a tool element 610 that may be assigned a higher priority record than the element 606. In this example, a user interface component 612 may include a window displaying an ultrasound image of the patient anatomy and may include a display characteristic that includes a display transparency for the window when overlaid on the tissue element 606 and the tool element 610. The display transparency may be fixed level of transparency such as 25% transparency, 50% transparency, 75% transparency, or any transparency level between 0 and 100% that may allow the viewing clinician to at least partially observe the obstructed tissue element 606 and tool element 610 underlying the user interface component 512. In alternative examples, the display transparency may be dynamic, growing more or less transparent, based upon changing illumination conditions, changes in the underlying elements, clinician selection, or other conditions in the field of view. The display characteristic for the user interface component 612 may also include a displayed outline 614 of edge demarcation of a structure in the field of view obstructed by the user interface component. For example, the user interface 612 may include a bolded outline, outline overlay, or otherwise accentuated border that marks the edge of the tool element 610 underlying the user interface component 612. In some examples, the displayed outline may only be displayed for portions of the user interface component that overlay an image element with a sufficiently high priority record (e.g., a priority record of 3 or greater on a 1-5 scale). In some examples, an overlay may be masked using an underlying structure (e.g., instrument outline) to reveal only pertinent features of the underlying structure. In alternative examples, the user interface component 612 may provide other types of information (e.g., alphanumeric labels, markers, shading) to indicate, describe, or mark the underlying image elements.

In some alternative examples, the display characteristic may be a display association or “pin” to an element in the image of the field of view or to a category or type of element in the image of the field of view (e.g., the category of surgically uninvolved tissue elements). In some examples, the category of element may be a type of structure distinguishable by a pixel or voxel type (e.g., pixels or voxels associated with the color or texture of body wall tissue) such that the user interface component may be pinned, tethered, attached, or otherwise displayed in association with the identified structure or type of pixel/voxel. In some examples, the clinician may assign or adjust the display characteristic, such as the display location. For example, the clinician may adjust the imaging system to provide a field of view image that includes a surgically uninvolved tissue or other noncritical tissue such as a body wall and may then assign a display location to a user interface component that pins or attaches the user interface component to the body wall.

In some examples, the display characteristic and/or the priority records may be dynamically updated if image analysis detects tissue movement, instrument movement, color change (e.g. bleeding) or other changes to the image of the field of view so that areas of change are unobstructed by the user interface components. In some examples, a clinician or other operator may be permitted or required to confirm changes to a display characteristic such as changes of location. In some examples, the display characteristic may allow a user to nudge the user interface component by moving an instrument visible in the field of view. In some examples, the nudging may be allowed while the medical system is in an instrument control or “following” mode of the medical system. In some examples, the display characteristics for a user interface component may be dependent on the type of display device. For example, the display location or other display characteristic for a user interface component and field of view image may have a certain set of values for a surgeon console head-in display system. The same field of view image and user interface component may have different display characteristics (e.g., display location) when displayed on a tablet or mobile device.

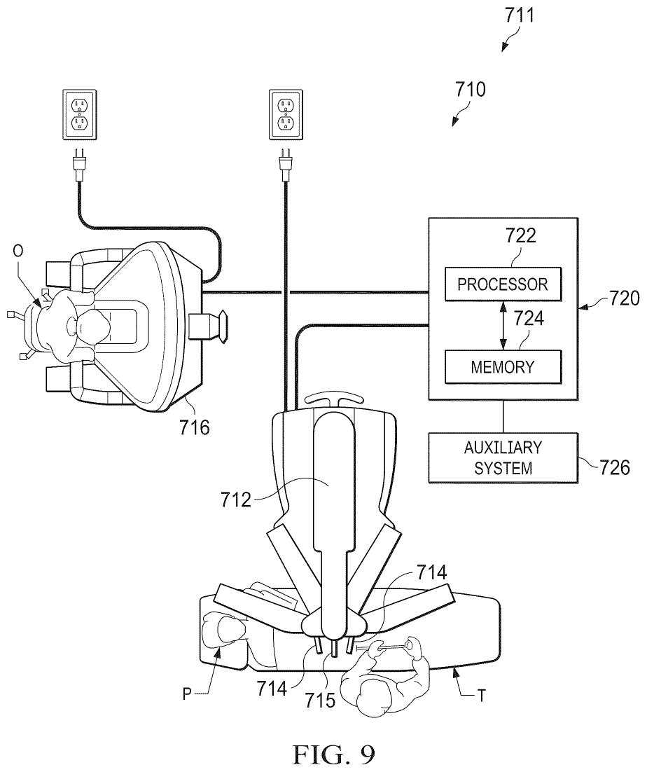

FIGS. 9-11 together provide an overview of a medical system 710 that may be used in, for example, medical procedures including diagnostic, therapeutic, or surgical procedures. The user interface display examples provided above may be used in the context of the medical system 710. The medical system 710 is located in a medical environment 711. The medical environment 711 is depicted as an operating room in FIG. 9. In other embodiments, the medical environment 711 may be an emergency room, a medical training environment, a medical laboratory, or some other type of environment in which any number of medical procedures or medical training procedures may take place. In still other embodiments, the medical environment 711 may include an operating room and a control area located outside of the operating room.

In one or more embodiments, the medical system 710 may be a robot-assisted medical system that is under the teleoperational control of an operator (e.g., a surgeon, a clinician, a physician, etc.). In alternative embodiments, the medical system 710 may be under the partial control of a computer programmed to perform the medical procedure or sub-procedure. In still other alternative embodiments, the medical system 710 may be a fully automated medical system that is under the full control of a computer programmed to perform the medical procedure or sub-procedure with the medical system 710. One example of the medical system 710 that may be used to implement the systems and techniques described in this disclosure is the da Vinci® Surgical System manufactured by Intuitive Surgical, Inc. of Sunnyvale, California.

As shown in FIG. 9, the medical system 710 generally includes an assembly 712, which may be mounted to or positioned near an operating table T on which a patient P is positioned. The assembly 712 may be referred to as a patient side cart, a surgical cart, or a surgical robot. In one or more embodiments, the assembly 712 may be a teleoperational assembly. The teleoperational assembly may be referred to as, for example, a teleoperational arm cart. A medical instrument system 714 and an endoscopic imaging system 715 are operably coupled to the assembly 712. An operator input system 716 allows an operator O or other type of clinician to view images of or representing the surgical site and to control the operation of the medical instrument system 714 and/or the endoscopic imaging system 715.

The medical instrument system 714 may comprise one or more medical instruments. In embodiments in which the medical instrument system 714 comprises a plurality of medical instruments, the plurality of medical instruments may include multiple of the same medical instrument and/or multiple different medical instruments. Similarly, the endoscopic imaging system 715 may comprise one or more endoscopes. In the case of a plurality of endoscopes, the plurality of endoscopes may include multiple of the same endoscope and/or multiple different endoscopes.

The operator input system 716 may be located at an operator's control console, which may be located in the same room as operating table T. In some embodiments, the operator O and the operator input system 716 may be located in a different room or a completely different building from the patient P. The operator input system 716 generally includes one or more control device(s) for controlling the medical instrument system 714. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some embodiments, the control device(s) will be provided with the same degrees of freedom as the medical instrument(s) of the medical instrument system 714 to provide the operator with telepresence, which is the perception that the control device(s) are integral with the instruments so that the operator has a strong sense of directly controlling instruments as if present at the surgical site. In other embodiments, the control device(s) may have more or fewer degrees of freedom than the associated medical instruments and still provide the operator with telepresence. In some embodiments, the control device(s) are manual input devices that are movable with six degrees of freedom, and which may also include an actuatable handle for actuating instruments (for example, for closing grasping jaw end effectors, applying an electrical potential to an electrode, delivering a medicinal treatment, and actuating other types of instruments).

The assembly 712 may support and manipulate the medical instrument system 714 while the operator O views the surgical site through the operator input system 716. An image of the surgical site may be obtained by the endoscopic imaging system 715, which may be manipulated by the assembly 712. The assembly 712 may comprise endoscopic imaging systems 715 and may similarly comprise multiple medical instrument systems 714 as well. The number of medical instrument systems 714 used at one time will generally depend on the diagnostic or surgical procedure to be performed and on space constraints within the operating room, among other factors. The assembly 712 may include a kinematic structure of one or more non-servo controlled links (e.g., one or more links that may be manually positioned and locked in place, generally referred to as a set-up structure) and a manipulator. When the manipulator takes the form of a teleoperational manipulator, the assembly 712 is a teleoperational assembly. The assembly 712 includes a plurality of motors that drive inputs on the medical instrument system 714. In an embodiment, these motors move in response to commands from a control system (e.g., control system 720). The motors include drive systems which when coupled to the medical instrument system 714 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of the medical instrument for grasping tissue in the jaws of a biopsy device or the like. Medical instruments of the medical instrument system 714 may include end effectors having a single working member such as a scalpel, a blunt blade, an optical fiber, or an electrode. Other end effectors may include, for example, forceps, graspers, scissors, or clip appliers.

The medical system 710 also includes a control system 720. The control system 720 includes at least one memory 724 and at least one processor 722 for effecting control between the medical instrument system 714, the operator input system 716, and other auxiliary systems 726 which may include, for example, imaging systems, audio systems, fluid delivery systems, display systems, illumination systems, steering control systems, irrigation systems, and/or suction systems. A clinician may circulate within the medical environment 711 and may access, for example, the assembly 712 during a set up procedure or view a display (e.g., display system 200) of the auxiliary system 726 from the patient bedside.

Though depicted as being external to the assembly 712 in FIG. 9, the control system 720 may, in some embodiments, be contained wholly within the assembly 712. The control system 720 also includes programmed instructions (e.g., stored on a non-transitory, computer-readable medium) to implement some or all of the methods described in accordance with aspects disclosed herein. While the control system 720 is shown as a single block in the simplified schematic of FIG. 9, the control system 720 may include two or more data processing circuits with one portion of the processing optionally being performed on or adjacent the assembly 712, another portion of the processing being performed at the operator input system 716, and the like.

Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one embodiment, the control system 720 supports wireless communication protocols such as Bluetooth, IrDA, HomeRF, IEEE 802.11, DECT, and Wireless Telemetry.

In some embodiments, control system 720 may include one or more servo controllers that receive force and/or torque feedback from the medical instrument system 714. Responsive to the feedback, the servo controllers transmit signals to the operator input system 716. The servo controller(s) may also transmit signals instructing assembly 712 to move the medical instrument system(s) 714 and/or endoscopic imaging system 715 which extend into an internal surgical site within the patient body via openings in the body. Any suitable conventional or specialized servo controller may be used. A servo controller may be separate from, or integrated with, assembly 712. In some embodiments, the servo controller and assembly 712 are provided as part of a teleoperational arm cart positioned adjacent to the patient's body.

The control system 720 can be coupled with the endoscopic imaging system 715 and can include a processor to process captured images for subsequent display, such as to an operator on the operator's control console, or on another suitable display located locally and/or remotely. For example, where a stereoscopic endoscope is used, the control system 720 can process the captured images to present the operator with coordinated stereo images of the surgical site. Such coordination can include alignment between the opposing images and can include adjusting the stereo working distance of the stereoscopic endoscope.

In alternative embodiments, the medical system 710 may include more than one assembly 712 and/or more than one operator input system 716. The exact number of assemblies 712 will depend on the surgical procedure and the space constraints within the operating room, among other factors. The operator input systems 716 may be collocated or they may be positioned in separate locations. Multiple operator input systems 716 allow more than one operator to control one or more assemblies 712 in various combinations. The medical system 710 may also be used to train and rehearse medical procedures.

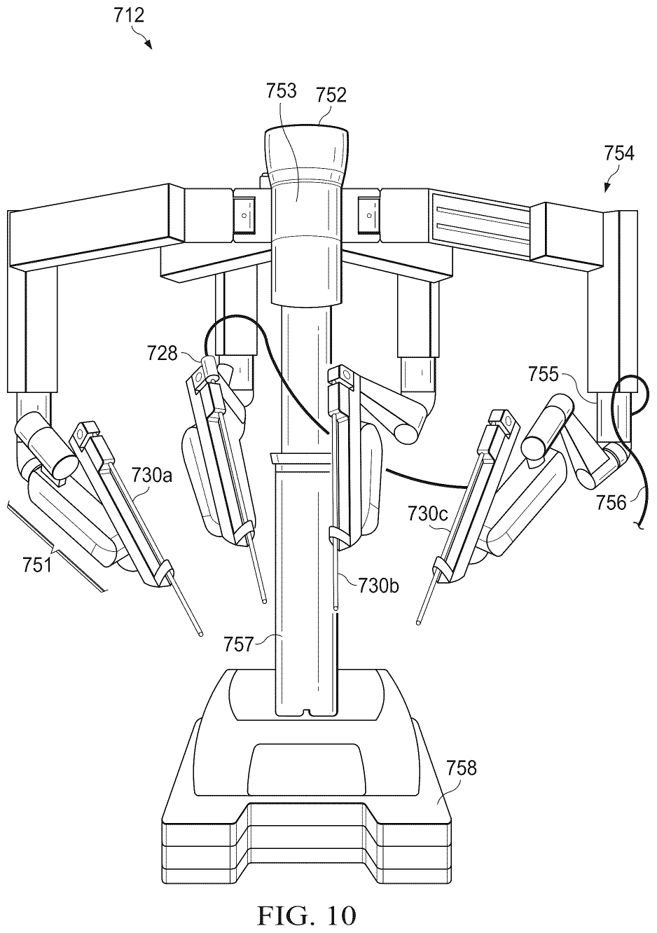

FIG. 10 is a perspective view of one embodiment of an assembly 712 which may be referred to as a patient side cart, surgical cart, teleoperational arm cart, manipulator assembly or surgical robot. The assembly 712 shown provides for the manipulation of three surgical tools 730a, 730b, and 730c (e.g., medical instrument systems 714) and an imaging device 728 (e.g., endoscopic imaging system 715), such as a stereoscopic endoscope used for the capture of images of the site of the procedure. The imaging device may transmit signals over a cable 756 to the control system 720. Manipulation is provided by teleoperative mechanisms having a number of joints. The imaging device 728 and the surgical tools 730a-c can be positioned and manipulated through incisions in the patient so that a kinematic remote center is maintained at the incision to minimize the size of the incision. Images of the surgical site can include images of the distal ends of the surgical tools 730a-c when they are positioned within the field of view of the imaging device 728.

The assembly 712 includes a drivable base 758. The drivable base 758 is connected to a telescoping column 757, which allows for adjustment of the height of arms 754. The arms 754 may include a rotating joint 755 that both rotates and moves up and down. Each of the arms 754 may be connected to an orienting platform 753. The arms 754 may be labeled to facilitate trouble shooting. For example, each of the arms 754 may be emblazoned with a different number, letter, symbol, other identifier, or combinations thereof. The orienting platform 753 may be capable of 760 degrees of rotation. The assembly 712 may also include a telescoping horizontal cantilever 752 for moving the orienting platform 753 in a horizontal direction.

In the present example, each of the arms 754 connects to a manipulator arm 751. The manipulator arms 751 may connect directly to a medical instrument, e.g., one of the surgical tools 730a-c. The manipulator arms 751 may be teleoperable. In some examples, the arms 754 connecting to the orienting platform 753 may not be teleoperable. Rather, such arms 754 may be positioned as desired before the operator O begins operation with the teleoperative components. Throughout a surgical procedure, medical instruments may be removed and replaced with other instruments such that instrument to arm associations may change during the procedure.

Endoscopic imaging systems (e.g., endoscopic imaging system 715 and imaging device 728) may be provided in a variety of configurations including rigid or flexible endoscopes. Rigid endoscopes include a rigid tube housing a relay lens system for transmitting an image from a distal end to a proximal end of the endoscope. Flexible endoscopes transmit images using one or more flexible optical fibers. Digital image-based endoscopes have a “chip on the tip” design in which a distal digital sensor such as a one or more charge-coupled device (CCD) or a complementary metal oxide semiconductor (CMOS) device store image data. Endoscopic imaging systems may provide two-or three-dimensional images to the viewer. Two-dimensional images may provide limited depth perception. Three-dimensional stereo endoscopic images may provide the viewer with more accurate depth perception. Stereo endoscopic instruments employ stereo cameras to capture stereo images of the patient anatomy. An endoscopic instrument may be a fully sterilizable assembly with the endoscope cable, handle and shaft all rigidly coupled and hermetically sealed.

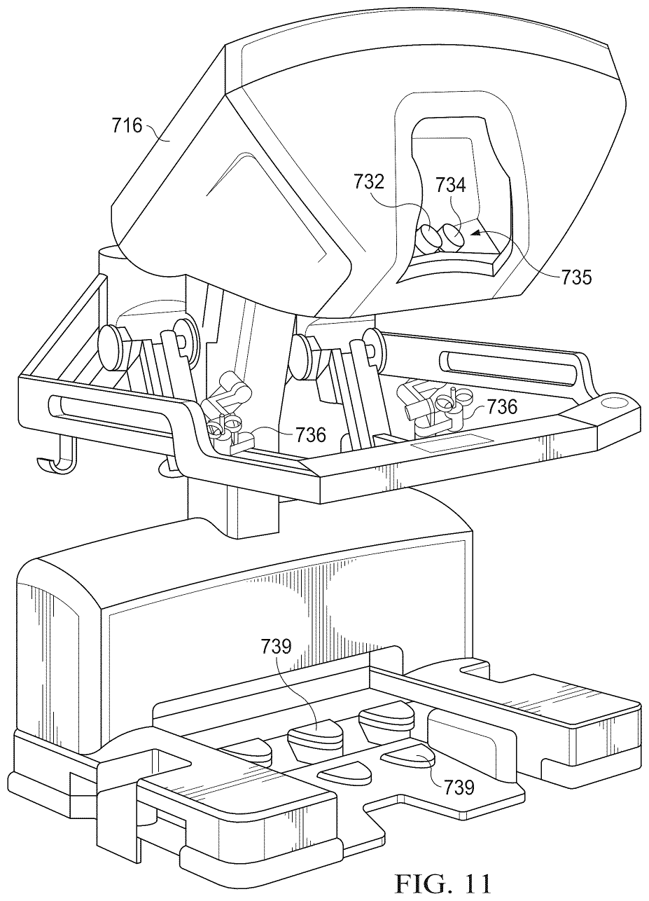

FIG. 11 is a perspective view of an embodiment of the operator input system 716 at the operator's control console. The operator input system 716 includes a display system with a left eye display 732 and a right eye display 734 for presenting the operator O with a coordinated stereo view of the surgical environment that enables depth perception. The left and right eye displays 732, 732 may be components of a display system 735 (e.g., the display system 200). In other embodiments, the display system 735 may include one or more other types of displays. The display system 735 may present images captured, for example, by the imaging system 715 to display the endoscopic field of view to the operator. The endoscopic field of view may be augmented by virtual or synthetic menus, indicators, and/or other graphical or textual information to provide additional information to the viewer.

The operator input system 716 further includes one or more input control devices 736, which in turn cause the assembly 712 to manipulate one or more instruments of the endoscopic imaging system 715 and/or medical instrument system 714. The input control devices 736 can provide the same degrees of freedom as their associated instruments to provide the operator O with telepresence, or the perception that the input control devices 736 are integral with said instruments so that the operator has a strong sense of directly controlling the instruments. To this end, position, force, and tactile feedback sensors (not shown) may be employed to transmit position, force, and tactile sensations from the medical instruments, e.g., surgical tools 730a-c, or imaging device 728, back to the operator's hands through the input control devices 736. Input control devices 739 are foot pedals that receive input from a user's foot. Aspects of the operator input system 716, the assembly 712, and the auxiliary systems 726 may be adjustable and customizable to meet the physical needs, skill level, or preferences of the operator O.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, systems, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately.

Various systems and portions of systems have been described in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A medical system comprising:
a display system for displaying a display area; and
a control system, wherein the control system includes a processing unit including one or more processors, and wherein the processing unit is configured to:
generate an image of a field of view for display in the display area;
generate a user interface component for display in the display area;
determine a priority record for an element in the image of the field of view;
determine a display characteristic for the user interface component based on the priority record of the element in the field of view; and
display the user interface component in accordance with the display characteristic in the display area overlayed on a displayed image of the field of view.

2. The medical system of claim 1, wherein the user interface component includes at least one of a menu, an alert, an indicator, a digital tool, or an auxiliary image.

3. The medical system of claim 1, wherein the user interface component includes modified image data based on an image analysis of image content.

4. The medical system of claim 1, wherein the priority record includes a ranking indicator of priority.

5. The medical system of claim 1, wherein the priority record is determined for an anatomic structure in the image of the field of view.

6. The medical system of claim 1, wherein the priority record is determined for a tool in the image of the field of view.

7. The medical system of claim 1, wherein the priority record is dynamic based on a detected activation in the field of view.

8. The medical system of claim 1, wherein determining the priority record for the element in the image of the field of view includes distinguishing a plurality of elements in the image of the field of view, including the element in the field of view.

9. The medical system of claim 8, wherein distinguishing the plurality of elements includes identifying a boundary between the plurality of elements.

10. The medical system of claim 8, wherein distinguishing the plurality of elements includes receiving a graphical segmentation of the image of the field of view.

11. The medical system of claim 8, wherein determining the priority record for the element in the image of the field of view includes receiving an indication of a selection from a priority menu for the element.

12. The medical system of claim 1, wherein determining a priority record for the element in the image of the field of view includes receiving a priority assignment.

13. The medical system of claim 1, wherein determining a priority record for the element in the image of the field of view includes receiving an indication of priority based on an image analysis of the image of the field of view.

14. The medical system of claim 13, wherein the image analysis includes an identification of a model priority record from a model co-registered with the image of the field of view.

15. The medical system of claim 1,
wherein the field of view includes a plurality of elements including the element, which is a first element, and a second element,
wherein the first element has a higher priority value than the second element, and
wherein displaying the user interface component with the display characteristic in the display area includes displaying the user interface component overlayed on the second element based on the first element's higher priority value.

16. The medical system of claim 1, wherein the processing unit is further configured to determine a procedure characteristic and wherein determining the display characteristic is further based on the procedure characteristic.

17. The medical system of claim 16, wherein the procedure characteristic is a type of procedure being performed in the field of view.

18. The medical system of claim 16, wherein the procedure characteristic is associated with an instrument in the image of the field of view.

19. The medical system of claim 1, wherein the display characteristic includes a display of a transparency level or blurring level of the user interface component.

20. The medical system of claim 1, wherein the display characteristic includes an outline of edge demarcation of a structure in the field of view obstructed by the user interface component.

21-36. (canceled)
